(19) United States
(12) Patent Application Publication
(10) Pub. No.: US 2024/0084559 A1
(43) Pub. Date: Mar. 14, 2024

(54) DISPENSING DEVICE AND HAND WASHING DEVICE

(71) Applicant: WOTA CORP., Chuo-ku, Tokyo (JP)

(72) Inventors: Kazunori KAWASHIMA, Tokyo (JP); Hiroki SUMA, Tokyo (JP); Ryo YAMADA, Tokyo (JP)

(73) Assignee: WOTA CORP., Chuo-ku, Tokyo (JP)

(21) Appl. No.: 18/517,095

(22) Filed: Nov. 22, 2023

Related U.S. Application Data
(63) Continuation of application No. PCT/JP2022/020506, filed on May 17, 2022.

(30) Foreign Application Priority Data
May 31, 2021 (JP) 2021-091240

(51) Int. Cl. E03B 1/04; A47K 5/12; E03C 1/182

(52) U.S. Cl. CPC E03B 1/042; A47K 5/12; E03C 1/182; E03B 2001/045; E03C 2201/40

(57) ABSTRACT

A dispensing device includes: a pump that discharges a liquid; a pipe through which a liquid discharged by the pump flows; a dispensing portion that is connected to the pipe and dispenses a liquid that flows through the pipe; a nozzle that houses the pipe, the dispensing portion being arranged therein in an opening portion; and an antenna that is provided inside the nozzle and transmits and receives radio signals.

### CROSS-REFERENCE TO RELATED APPLICATIONS

This application is based upon and claims the benefit of priority from the prior Japanese Patent Applications No. 2021-091240, filed May 31, 2021, and from PCT Patent Applications No. PCT/JP2022/20506, filed May 17, 2022, the entire contents of which are incorporated herein by reference.

### FIELD

Embodiments described herein relate generally to a dispensing device and a hand washing device.

### BACKGROUND

Technology is already known in which communication means is provided in a water purifier or the like that is attached to a faucet and transmits information indicating a state inside the water purifier to outside by wireless communication.

In a case where a water purifier is attached to the outside of a faucet, communication means may be provided to the water purifier itself, and therefore it is not necessary to provide communication means in the faucet. On the other hand, in a case where a water purification mechanism is provided at a position that cannot be visually recognized from the outside, where the communication means is provided is aesthetically important for a dispensing device.

An object of the present disclosure is to provide a dispensing device which can maintain an elegant appearance while being equipped with communication means for communicating with an external device.

### BRIEF DESCRIPTION OF DRAWINGS

FIG. 1 is a block diagram of a hand washing device in which a dispensing device is mounted.

FIG. 2 is an external perspective view of the hand washing device in which the dispensing device is mounted.

FIG. 3 is an external perspective view from the rear of the hand washing device illustrated in FIG. 1.

FIG. 4 is a top view of the hand washing device illustrated in FIG. 1.

FIG. 5 is a block diagram for describing each unit of the hand washing device illustrated in FIG. 1.

FIG. 6 is an external view of the dispensing device.

FIG. 7 is a cross-sectional view of the dispenser illustrated in FIG. 6.

FIG. 8 is a front view of an externally attached module shown in FIG. 2.

FIG. 9A is explanatory drawing illustrating control processing for a circulation unit.

FIG. 9B is explanatory drawing illustrating control processing for a circulation unit.

FIG. 9C is explanatory drawing illustrating control processing for a circulation unit.

FIG. 10 is an explanatory drawing illustrating control processing for a hand-washing indicator.

FIG. 11A is view for describing display mode of the hand-washing indicator.

FIG. 11B is view for describing display mode of the hand-washing indicator.

FIG. 11C is view for describing display mode of the hand-washing indicator.

FIG. 11D is view for describing display mode of the hand-washing indicator.

FIG. 12 is a block diagram of a hand washing device in which a dispensing device according to a modification is mounted.

FIG. 13 is a block diagram for describing each unit of the hand washing device illustrated in FIG. 12.

FIG. 14 is a cross-sectional view of the dispensing device according to the modification.

FIG. 15 is a cross-sectional view of a dispensing device according to another modification.

### DETAILED DESCRIPTION

In general, according to one embodiment, a dispensing device includes: a pump that discharges a liquid; a pipe through which a liquid discharged by the pump flows; a dispensing portion that is connected to the pipe and dispenses a liquid that flows through the pipe; a nozzle that houses the pipe, the dispensing portion being arranged therein in an opening portion; and an antenna that is provided inside the nozzle and transmits and receives radio signals.

Hereunder, one embodiment of the present disclosure will be described in detail based on the accompanying drawings. Note that, in the drawings for describing the embodiment, in principle, the same reference symbols are assigned to the same constituent elements, and repeated description thereof is omitted.

### Overview

A hand washing device 1 according to the present embodiment purifies and circulates drained water after washing hands, to thereby realize hygienic hand washing without receiving a supply of water from a mains water supply. A dispensing device according to the present embodiment is used as a part of this type of hand washing device 1. The dispensing device dispenses a liquid which is stored in a tank of the hand washing device 1. Water or a pharmaceutical agent may be mentioned as examples of a liquid that the dispensing device dispenses. An antenna for enabling the hand washing device 1 to communicate with an external device is provided inside the dispensing device.

In the present embodiment, a dispenser that dispenses a pharmaceutical agent for maintaining skin hygiene is taken as one example of the dispensing device. In the following description, first, a configuration of the hand washing device 1 will be described, and thereafter a function of the antenna which the dispenser has will be described in detail.

### Overall Configuration

The overall configuration of the hand washing device 1 according to the present embodiment will now be described.

The hand washing device 1 can be installed at various places such as at an indoor location or an outdoor location. As an indoor location, for example, the hand washing device 1 is installed near an entrance of a building, in office space that a business person occupies in a building, near an entrance of a store, or the like. For example, in a case where a building is a facility used for lodging, exercise, entertainment, or the like, and it is required to check-in in order to utilize the facility, the hand washing device 1 is installed at a place where people check-in. For example, it is assumed that users wash their hands using the hand washing device 1 before or after writing in predetermined matters with a shared writing implement in order to check-in.

The hand washing device 1 has a function of sterilizing articles carried by users who wash their hands. Examples of articles that the hand washing device 1 sterilizes that may be mentioned include smartphones, tablet terminals, and the like. Note that, the hand washing device 1 may sterilize articles other than these. The sterilization of articles by the hand washing device 1 is performed simultaneously with the action of washing hands when, for example, a person who visits a restaurant washes their hands when entering the restaurant or the like.

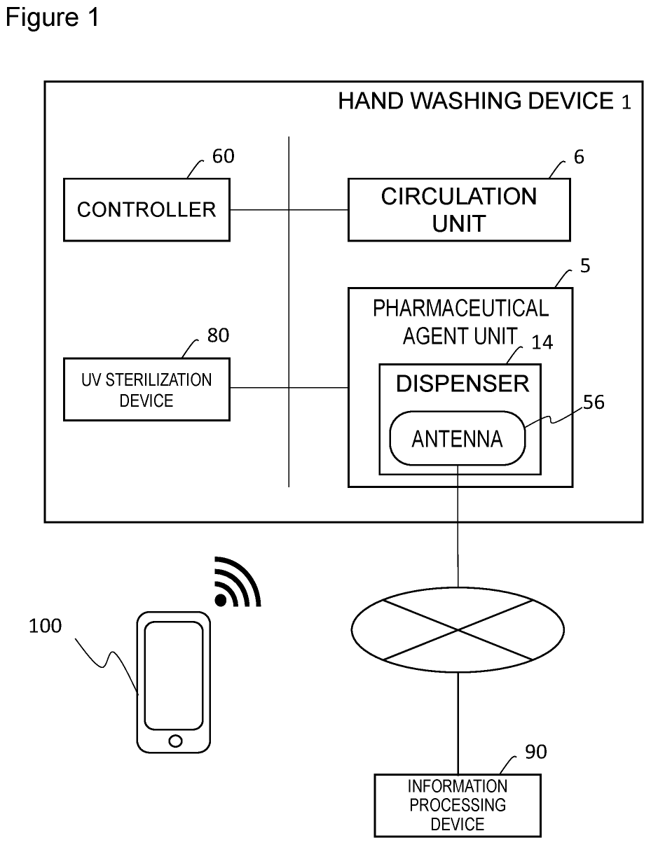

FIG. 1 is a block diagram representing a functional configuration of the hand washing device 1 of the present embodiment. As illustrated in FIG. 1, the hand washing device 1 includes a controller 60, a UV sterilization device 80, a circulation unit 6, and a pharmaceutical agent unit 5.

The controller 60 has a function of controlling each unit of the hand washing device 1.

The controller 60 is realized by a processor reading out a program stored in a storage and expanding the program on a memory, and executing commands included in the expanded program. The processor is hardware for executing a set of commands described in a program, and is composed of an arithmetic unit, a register, a peripheral circuit, and the like. The storage is a storage device for storing data, such as a flash memory or a HDD (hard disc drive). The memory is for temporarily storing programs and also data to be processed by the programs and the like, and for example is a volatile memory such as a DRAM (dynamic random access memory).

The UV sterilization device 80 has a function of irradiating ultraviolet rays at an article of a person washing their hands with washing water, to thereby sterilize a surface of the article. A configuration of the UV sterilization device 80 will be described later.

The circulation unit 6 has a function of purifying drained water which the user have used to wash their hands, and then circulating the purified water. A configuration of the circulation unit 6 will be described later.

The pharmaceutical agent unit 5 has a function of dispensing a pharmaceutical agent for maintaining skin hygiene to a user who utilizes the hand washing device 1. Examples of pharmaceutical agents for maintaining skin hygiene include cleansing agents for cleaning the skin (for example, a cleanser such as soapy water), and liquids or hand lotions or the like having a bactericidal action (for example, a disinfectant containing an ingredient such as alcohol). The pharmaceutical agent unit 5 has a dispenser 14 that dispenses the pharmaceutical agent. The dispenser 14 has an antenna 56 inside the dispenser 14.

The antenna 56 is a device for allowing the hand washing device 1 to transmit radio waves and to receive radio waves which arrive from outside. Configurations of the pharmaceutical agent unit 5, the dispenser 14, and the antenna 56 are described later.

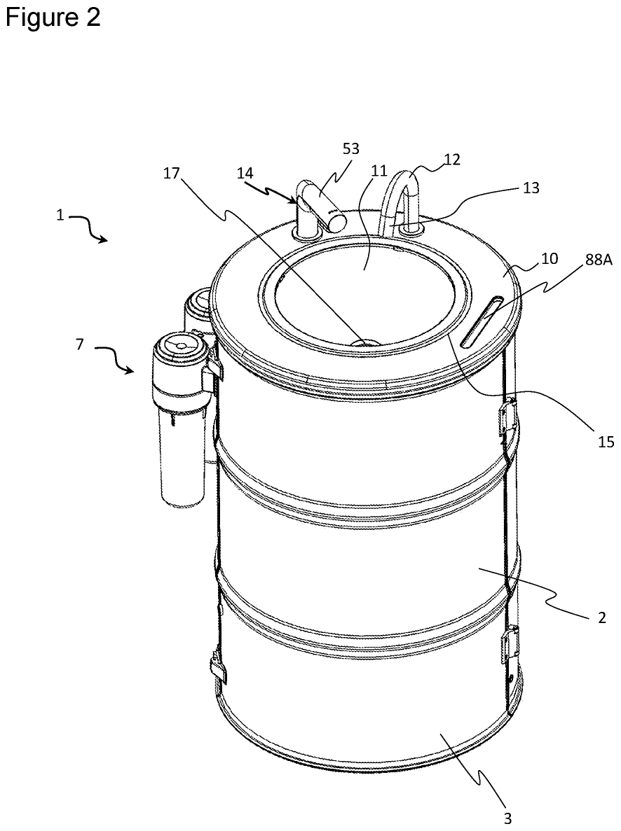

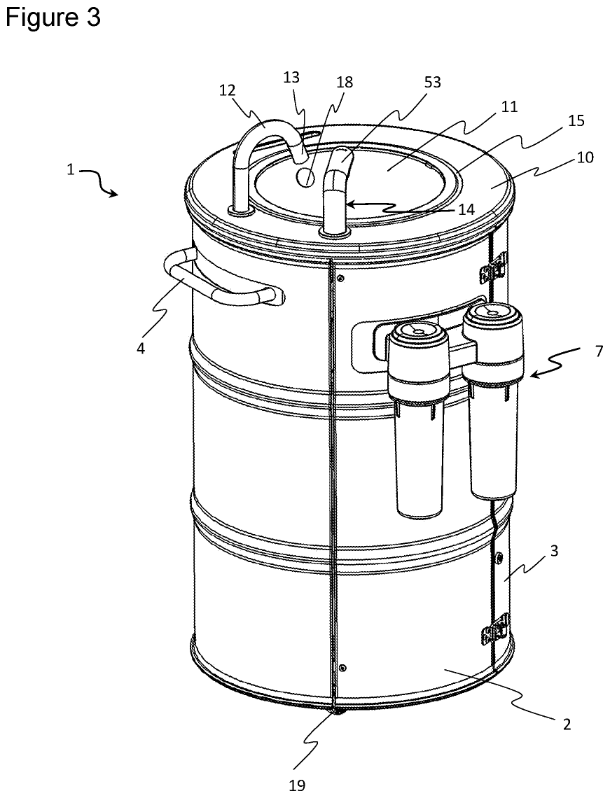

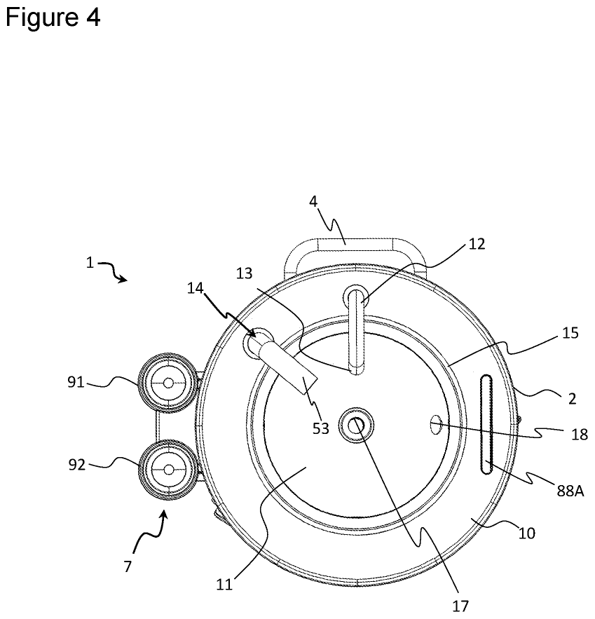

FIG. 2 and FIG. 3 are external perspective views of the hand washing device 1 of the present embodiment. FIG. 4 is a top view of the hand washing device 1. Note that, in the following description, a side on which a user who uses the hand washing device 1 stands is referred to as the front of the hand washing device 1, and an opposite side thereto is referred to as the rear of the hand washing device.

As illustrated in FIG. 2, the hand washing device 1 includes a casing 2 and an externally attached module 7.

### <Configuration of Casing 2>

The casing 2 is formed in a cylindrical shape. The casing 2 is made by, for example, processing a metallic drum can which was made using stainless steel or steel as a material. A top plate 10 is provided at an upper part of the casing 2. An installation hole that penetrates the top plate 10 is formed in a central part of the top plate 10.

A door 3 for accessing a circulation unit 6 provided inside the casing 2 is provided in an outer peripheral surface of the casing 2. A manager of the hand washing device 1 can perform maintenance of the circulation unit 6 in a state in which the door 3 is open.

An entire structure of the circulation unit 6 is not provided inside the casing 2, and at least one part of the circulation unit 6 is arranged outside the casing 2 as a part of the externally attached module 7.

As illustrated in FIG. 3, a handle 4 extending in a lateral direction is provided on the rear of the casing 2.

A plurality of wheels 19 are provided on a bottom surface of the casing 2. The user can move the casing 2 by moving the wheels 19 in a state in which the user grips the handle 4.

As illustrated in FIG. 4, a wash-hand basin 11 is provided in the casing 2. The wash-hand basin 11 is provided in one part of the casing 2, and has a circular bowl shape in top view. In the illustrated example, the wash-hand basin 11 is arranged by being fitted into the installation hole in the top plate 10 provided in the casing 2.

A drainage outlet 17 for draining washing water spouted into the basin is formed in a bottom of the wash-hand basin 11.

An overflow hole 18 is formed in an inner peripheral surface of the wash-hand basin 11 to prevent washing water overflowing onto the top plate 10 by draining the washing water when a level of washing water in the basin exceeds a predetermined water level.

A faucet 12 is provided in the casing 2. A spout 13 for spouting washing water is formed at a tip of the faucet 12. An infrared sensor 23 (see FIG. 4) is provided in the faucet 12.

The infrared sensor 23 is provided alongside the spout 13 at the tip of the faucet 12. When an object is detected by the infrared sensor 23, washing water is spouted from the spout 13 of the faucet 12.

Note that, a position at which the infrared sensor 23 is provided in the faucet 12 is not limited to the tip, and can be changed arbitrarily.

As illustrated in FIG. 2, a base of the faucet 12 is attached to the top plate 10. The faucet 12 is constructed so as to extend upward from the base towards the tip, and is curved at a central part so that the spout 13 that is located at the tip faces downward.

As illustrated in FIG. 2, a hand-washing indicator 15 that is an indicator light showing a rough estimate of a hand-washing time period of the user is provided on an upper face of the casing 2. The hand-washing indicator 15 is arranged on an upper face of the top plate 10 of the casing 2.

The hand-washing indicator 15 is arranged outside the wash-hand basin 11 on the upper face of the top plate 10, and is formed so as to surround the wash-hand basin 11 at an upper edge of the wash-hand basin 11.

The hand-washing indicator 15 is composed of, for example, a plurality of LED lights. The plurality of LED lights are arranged, for example, at intervals in a circumferential direction at the upper edge of the wash-hand basin 11.

In the present embodiment the hand-washing indicator 15 is composed of 30 LED lights.

### <Configuration of Pharmaceutical Agent Unit 5>

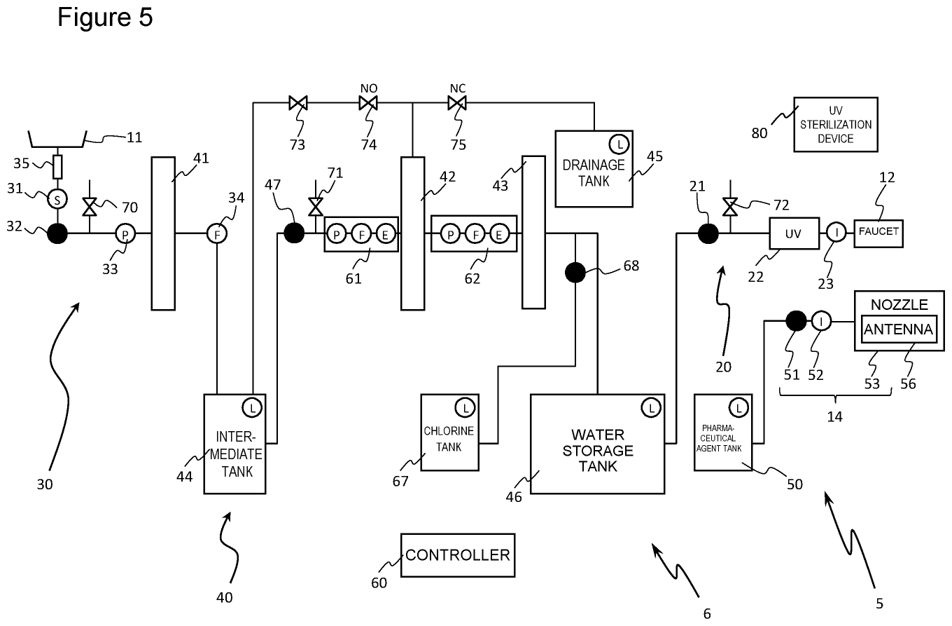

FIG. 5 is a block diagram for describing each unit of the hand washing device 1.

As illustrated in FIG. 5, the pharmaceutical agent unit 5 includes a pharmaceutical agent tank 50 and the dispenser 14. The dispenser 14 includes a pharmaceutical agent pump 51, an infrared sensor 52, a nozzle 53, and the antenna 56.

The pharmaceutical agent tank 50 and the pharmaceutical agent pump 51 are provided inside the casing 2.

The infrared sensor 52 and the antenna 56 are provided inside the nozzle 53.

The pharmaceutical agent tank 50 is a tank for storing a pharmaceutical agent. A liquid level sensor is arranged in the pharmaceutical agent tank 50. The liquid level sensor detects a level of the pharmaceutical agent stored inside the pharmaceutical agent tank 50. When the level of the pharmaceutical agent drops below a predetermined value, the pharmaceutical agent is replenished.

The pharmaceutical agent pump 51 is arranged at a subsequent stage to the pharmaceutical agent tank 50. The pharmaceutical agent pump 51 is operated under the control of the controller 60, and sends a pharmaceutical agent that is stored in the pharmaceutical agent tank 50 to the nozzle 53 of the dispenser 14.

Specifically, the controller 60 actuates the pharmaceutical agent pump 51 in response to the detection of an object by the infrared sensor 52 of the dispenser 14. For example, the controller 60 causes the pharmaceutical agent pump 51 to operate during a period in which an object is detected by the infrared sensor 52. When an object is no longer detected by the infrared sensor 52, the controller 60 stops the pharmaceutical agent pump 51.

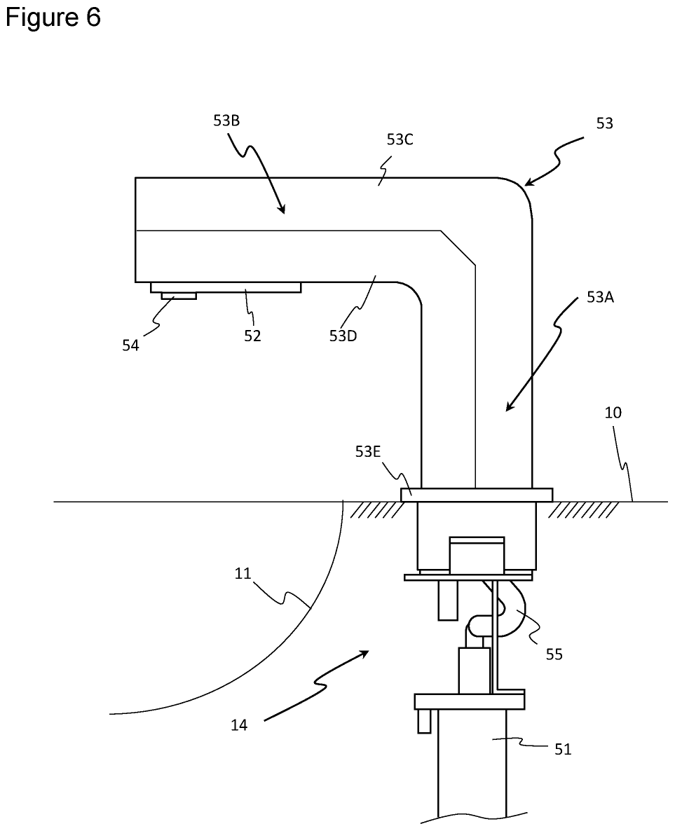

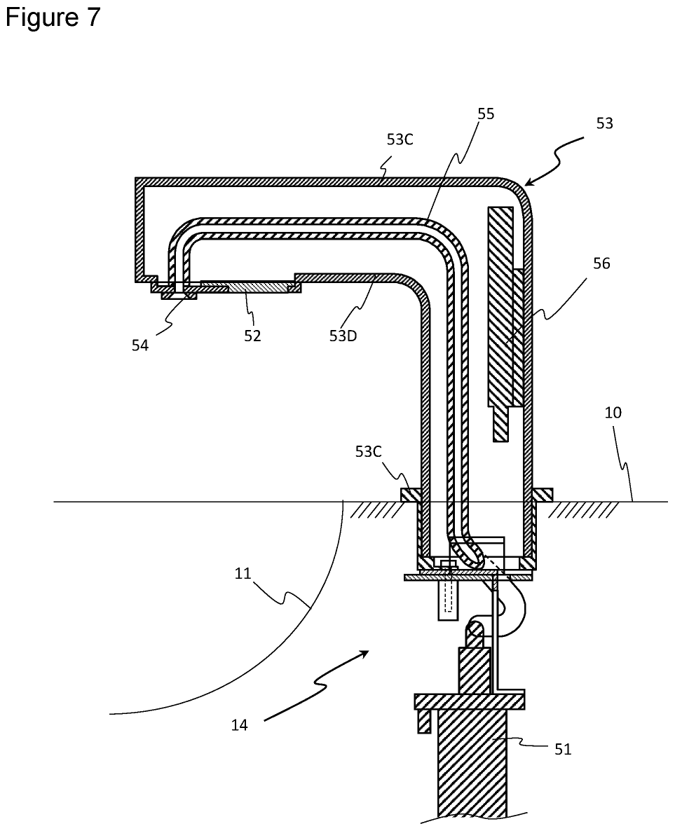

FIG. 6 shows an external view of the dispenser 14. FIG. 7 shows a cross-sectional view of the dispenser 14. In FIG. 6 and FIG. 7, to facilitate the description, a configuration of a portion of the dispenser 14 which is located inside the casing 2 is also illustrated.

As illustrated in FIG. 6 and FIG. 7, the nozzle 53 is a pipe-shaped member that includes a channel for a pharmaceutical agent therein. The nozzle 53 includes a base 53A that extends upward from the top plate 10 of the casing 2, and an arm portion 53B that extends in a horizontal direction from the upper end of the base 53A.

A flange 53E that protrudes outward in the horizontal direction is formed at a lower part of the base 53A. A portion of the base 53A that is located below the flange 53E is inserted into a mounting hole provided in the top plate 10. When the nozzle 53 is inserted into the mounting hole, the undersurface of the flange 53E contacts the upper surface of the top plate 10.

An opening is formed at the tip of the arm portion 53B. A dispensing portion 54 is arranged in the opening. The dispensing portion 54 is a portion that dispenses the pharmaceutical agent that flows through a pipe 55 housed inside the nozzle 53, toward the inside of the wash-hand basin 11.

The nozzle 53 is formed by combining an upper case 53C and a bottom case 53D. The upper case 53C is a member constituting the upper side of the nozzle 53, and the bottom case 53D is a member constituting the lower side of the nozzle 53.

The upper case 53C and the bottom case 53D are made of a synthetic resin material.

As illustrated in FIG. 7, the pipe 55 and the antenna 56 are housed inside the nozzle 53. Further, in the vicinity of the tip of the nozzle 53, the infrared sensor 52 is provided at a position where it is possible to detect one or more fingers that approach the inside of the wash-hand basin 11.

One end of the pipe 55 is connected to the pharmaceutical agent pump 51, and the other end of the pipe 55 is connected to the dispensing portion 54. Inside the casing 2, the pharmaceutical agent pump 51 is arranged at a position below the nozzle 53.

The pipe 55 allows the pharmaceutical agent sent from the pharmaceutical agent pump 51 to flow therein. The pipe 55 forms a channel for the pharmaceutical agent inside the nozzle 53 so as to follow the shape of the nozzle 53.

The antenna 56 is arranged inside the nozzle 53 along a direction in which the pipe 55 is extended. The antenna 56 is arranged inside a portion of the upper case 53C of the nozzle 53 that constitutes one part of the base 53A. The antenna 56 transmits radio signals relating to various types of information to an external information processing device, and receives such radio signals from the information processing device.

### <Communication by Antenna 56>

The antenna 56 is, for example, an antenna that is capable of receiving radio waves in accordance with at least any one communication standard. Further, the antenna 56 is an antenna that is capable of transmitting radio waves in accordance with at least any one communication standard.

For example, the antenna 56 may be compatible with a communication service provided by a telecommunications carrier. In such a case, for example, a communication function realized by the controller 60 of the hand washing device 1 is compatible with the communication service that the telecommunications carrier provides. By this means it is possible for the hand washing device 1 to be connected via the antenna 56 to a network which the telecommunications carrier provides.

Further, for example, the antenna 56 may be compatible with a wireless LAN such as Wi-Fi (registered trademark). In such a case, for example, a communication function realized by the controller 60 of the hand washing device 1 performs communication processing that corresponds to the wireless LAN. By this means it is possible for the hand washing device 1 to be connected via the antenna 56 to a network formed by the wireless LAN.

Further, for example, the antenna 56 may be compatible with a short-range wireless communication technology such as Bluetooth (registered trademark), ZigBee (registered trademark), or BLE (Bluetooth Low Energy). In such a case, for example, a communication function realized by the controller 60 of the hand washing device 1 performs communication processing that corresponds to the short-range wireless communication such as Bluetooth (registered trademark), ZigBee (registered trademark), or BLE (Bluetooth Low Energy). By this means it is possible for the hand washing device 1 to be communicably connected via the antenna 56 to a portable item (a communication device such as a smartphone or a smartwatch) that supports the short-range wireless communication.

In addition, for example, the antenna 56 may be compatible with a short-range wireless communication technology such as NFC (near field communication). In such a case, for example, a communication function realized by the controller 60 of the hand washing device 1 performs communication processing that corresponds to the short-range wireless communication such as NFC. By this means it is possible for the hand washing device 1 to read out data from a portable item that supports the short-range wireless communication via the antenna 56.

For example, the following data may be mentioned as data which is transmitted or as data which is received by the hand washing device 1 via the antenna 56.
Data relating to hand washing
Data relating to maintenance
Data input from portable item
Data transmitted to portable item

The data relating to hand washing includes, for example, the following data.
(A) Data pertaining to the usage history of the hand washing device 1
   (1) Dates and times hand washing device 1 was used
   (2) Information relating to operation of hand washing device 1
(B) Data obtained by monitoring hand washing device 1 that is acquired from monitoring device
(C) Various kinds of aggregated data created based on detected data
(D) Other data

The monitoring device is a device that monitors the area surrounding the hand washing device 1. The monitoring device is installed at a position at which it is possible to monitor the area surrounding the hand washing device 1. For example, the monitoring device may be a camera or the like arranged in a space (a lobby or an entrance of a building or the like) where the hand washing device 1 is installed. In a case where the hand washing device 1 is installed in a space that is utilized by unspecified users, the monitoring device may be a surveillance camera or the like already installed in the space, or may be a device that is installed to match the installation location of the hand washing device 1. The monitoring device is realized by an image pickup device such as a camera, or a sensor such as a motion sensor or an infrared sensor. The monitoring device transmits acquired monitoring data, for example, image data or sensor data, to the hand washing device 1.

The data relating to maintenance includes, for example, the following data.

### (A) Parameters for Driving Various Kinds of Hardware Constituting Hand Washing Device

Parameters Relating to Driving a Pump
(1) Driving values (voltage, current) for pump, cumulative driving time, pump maintenance record
(2) Service life of pump

Parameters Relating to Tank Operation Control
(1) Capacity, material, and service life of tank
(2) Fluctuations in capacity of tank
(3) Cumulative driving time and maintenance record of tank

Parameters Relating to Valve Operation Control
(1) Driving values (voltage, current) for valve, cumulative driving time, operating time/timing
(2) Service life of valve

Parameters Relating to Filter Operation Control
(1) Cleaning capacity of filter
(2) Number of allowable uses of filter

Parameters Relating to Operation Control of UV Sterilization Device
(1) Driving values (voltage, current) and cumulative driving time of light source and motor, maintenance record of sterilization device
(2) Service life of light source and motor

### (B) Sensing Results

Sensing Results of Various Sensors Capable of Sensing in Water (for Example, Each of the Following Items can be Sensed by a Sensor)
(1) Flow rate, pressure, water level, temperature
(2) pH, electric conductivity, oxidation-reduction potential, alkalinity, ion concentration, hardness
(3) Turbidity, chromaticity, viscosity, dissolved oxygen
(4) Odor, ammonia nitrogen, nitrate nitrogen, nitrite nitrogen, total nitrogen, residual chlorine, total phosphorus, total organic carbon, total inorganic carbon, total trihalomethane
(5) detection result of microbial sensor, chemical oxygen demand, biological oxygen demand
(6) Cyanogen, mercury, oil content, surfactants
(7) detection result of optical sensor, detection result of TDS (total dissolved solids) sensor
(8) mass spectrometry result, fine particles, zeta potential, surface potential

Image Data
(1) Monitoring image data of hand washing
(2) Monitoring image data of tank
(3) Monitoring image data of filter
(4) Monitoring image data of piping

### (C) Other Information

Environmental Information
(1) Weather, atmospheric pressure, temperature, humidity
(2) Information indicating location of environment (installation location, facility name)
(3) Information relating to municipality where hand washing device is installed (economic policies, disaster contingency plan, sanitation measures)

Data Pertaining to Users
(1) User attributes (gender, nationality, place of residence, occupation)
(2) Usage history of users (hand washing time, frequency)

Data Pertaining to Manager
(1) Manager attributes (gender, nationality, place of residence, industry, contact information)
(2) Management history of manager (maintenance history, consumable items purchase history)
(3) Management system of manager (operation standards, work schedule)

The data input from a portable item of a user includes, for example, data which a user inputs when utilizing the hand washing device 1. The data which a user inputs includes, for example, the following data.
User attributes (gender, nationality, place of residence, occupation)
Data such as evaluation of hand washing device 1 from user

Specifically, the antenna 56 receives information pertaining to the user that is transmitted from a smartphone that has been brought close to the dispenser 14, in accordance with, for example, short-range wireless communication such as NFC.

The data transmitted to a portable item of the user includes, for example, data to inform the user of the existence of the hand washing device 1 and to urge the user to use the hand washing device 1.

Specifically, the antenna 56 transmits data to inform the user of the existence of the hand washing device 1 to a user terminal 100 which the user carries with them, in accordance with, for example, short-range wireless communication such as Bluetooth (registered trademark) or BLE (Bluetooth Low Energy).

More specifically, the antenna 56 transmits a beacon signal which is capable of reaching up to a predetermined range and which includes information for identifying the hand washing device 1. The user terminal 100 has a function of receiving the beacon signal transmitted by the antenna 56. Further, for example, an application for utilizing the hand washing device 1 is stored in the user terminal 100.

When the user terminal 100 enters within a range that the relevant beacon signal reaches, the user terminal 100 receives the beacon signal. Upon receiving the beacon signal, the user terminal 100 activates an application corresponding to the beacon signal, that is, the application for utilizing the hand washing device 1. By means of a function realized by the application, the user terminal 100, for example, accesses an external server, and requests the server information that conveys the existence of the hand washing device 1 corresponding to the beacon signal, and information to the effect of urging use of the hand washing device 1. By means of this request, the server ascertains the location of the user who uses the user terminal 100. Upon being requested information conveying the existence of the hand washing device 1 and information to the effect of urging use of the hand washing device 1, the server transmits the requested information to the user terminal 100 that is the source of the request. The user terminal 100 receives the information conveying the existence of the hand washing device 1 and the information to the effect of urging use of the hand washing device 1, and displays the received information on a display.

### <Configuration of Circulation Unit 6>

As illustrated in FIG. 5, the pharmaceutical agent unit 5 that supplies a pharmaceutical agent, and the circulation unit 6 that purifies and circulates washing water are provided inside the casing 2. The pharmaceutical agent unit 5 and the circulation unit 6 are controlled by the controller 60.

As illustrated in FIG. 5, the circulation unit 6 includes at least a spout unit 20, a drainage unit 30, and a purification unit 40.

In the circulation unit 6, the spout unit 20 has a function of causing water purified by the purification unit 40 to spout from the spout 13 of the faucet 12.

The spout unit 20 mainly includes a spout pump 21, a UV sterilization section 22, and the infrared sensor 23.

The spout pump 21 is arranged at a subsequent stage to a water storage tank 46 that is provided in the purification unit 40. The spout pump 21 is operated under the control of the controller 60 to send water stored in the water storage tank 46 to the UV sterilization section 22.

Specifically, the controller 60 actuates the spout pump 21 in response to detection of an object by the infrared sensor 23. For example, the controller 60 causes the spout pump 21 to operate during a period in which an object is detected by the infrared sensor 23. When the object is no longer detected by the infrared sensor 23, the controller 60 stops the spout pump 21.

The UV sterilization section 22 is arranged between the spout pump 21 and the faucet 12. The UV sterilization section 22 irradiates ultraviolet rays at water sent from the spout pump 21 to subject the water to a sterilization treatment. Water that has passed through the UV sterilization section 22 is spouted from the spout 13 of the faucet 12 as washing water.

An air vent 72 for removing air in the water sent from the spout pump 21 is provided between the spout pump 21 and the UV sterilization section 22.

In the circulation unit 6, the drainage unit 30 has a function of draining washing water spouted from the faucet 12 toward the wash-hand basin 11.

The drainage unit 30 mainly includes a trap 35, a capacitance sensor 31, and a drainage pump 32.

The trap 35 is provided in a pipe that drains washing water from the wash-hand basin 11. The trap 35 prevents, for example, a backward flow of a bad odor or gas or the like, and also prevents a foreign matter which entered from the drainage outlet 17 from reaching the purification unit 40.

The drainage pump 32 is arranged at a subsequent stage to the trap 35. The drainage pump 32 is operated under control of the controller 60, and sends water that passed through the trap 35 to a pretreatment filter 41 provided in the purification unit 40.

Specifically, the controller 60 actuates the drainage pump 32 in response to the detection of water by the capacitance sensor 31. For example, the controller 60 causes the drainage pump 32 to operate during a period in which water is detected by the capacitance sensor 31. When water is no longer detected by the capacitance sensor 31, the controller 60 stops the drainage pump 32.

The capacitance sensor 31 is arranged between the trap 35 and the drainage pump 32. The capacitance sensor 31 detects the capacitance inside the drainage pipe. By this means, water drained from the wash-hand basin 11 and supplied through the trap 35 is detected. Note that, a sensor for detecting the supply of water is not limited to the capacitance sensor 31. The supply of water may be detected by referring to a different sensing result.

An air vent 70 for removing air in the water sent from the drainage pump 32 is provided between the drainage pump 32 and the purification unit 40.

In the circulation unit 6, the purification unit 40 has a function of purifying the water supplied from the drainage unit 30.

The purification unit 40 mainly includes the pretreatment filter 41, a reverse osmosis membrane 42, a post-treatment filter 43, an intermediate tank 44, a drainage tank 45, the water storage tank 46, and a membrane filtration pump 47.

The pretreatment filter 41 is arranged at a subsequent stage to the drainage pump 32. The pretreatment filter 41 subjects the water sent from the drainage pump 32 to a pretreatment that removes a solid content, water polluting components, low-molecular-weight compound surfactants, carbonic acid components (detergent components), and the like.

Although in the present embodiment an activated carbon filter is adopted as the pretreatment filter 41, the present disclosure is not limited thereto. For example, at least any one of a string wound filter, a sediment filter, an MF (microfiltration membrane), a UF (ultrafiltration membrane), an NF (nano-filtration membrane), a ceramic filter, an ion exchange filter, and a metal membrane may be selected. In the present embodiment, the pretreatment filter 41 is arranged outside the casing 2 as a part of the externally attached module 7.

A pressure sensor 33 is arranged at a preceding stage to the pretreatment filter 41. The pressure sensor 33 detects a pressure of water supplied to the pretreatment filter 41.

A flow rate sensor 34 is arranged at a subsequent stage to the pretreatment filter 41. The flow rate sensor 34 detects a flow rate of water pretreated by the pretreatment filter 41.

The intermediate tank 44 is arranged at a subsequent stage to the pretreatment filter 41. The intermediate tank 44 is a tank for storing supplied water. Water subjected to the pretreatment by the pretreatment filter 41 and concentrated water separated by the reverse osmosis membrane 42 and passed through a two-way solenoid valve 74 flow into the intermediate tank 44. The intermediate tank 44 stores the water that flows in from these two systems.

A water level sensor is arranged in the intermediate tank 44. The water level sensor detects a level of the water stored inside the intermediate tank 44.

The membrane filtration pump 47 is arranged between the intermediate tank 44 and the reverse osmosis membrane 42.

The membrane filtration pump 47 is operated under the control of the control unit 60, and pressurizes the water stored in the intermediate tank 44 to a preset pressure and supplies the water to the reverse osmosis membrane 42. Note that, the preset pressure is, for example, a pressure that is at least higher than the osmotic pressure.

The reverse osmosis membrane 42 separates the water pressurized to a high pressure and supplied by the membrane filtration pump 47 into permeated water from which dissolved components have been removed, and concentrated water in which dissolved components have been concentrated. The reverse osmosis membrane 42 is realized, for example, by a spiral reverse osmosis membrane.

When the two-way solenoid valve 74 is open, the concentrated water separated by the reverse osmosis membrane 42 is discharged to the intermediate tank 44 through the two-way solenoid valve 74 and a pressure regulating valve 73. Further, when a two-way solenoid valve 75 is open, the concentrated water separated by the reverse osmosis membrane 42 is discharged to the drainage tank 45 through the two-way solenoid valve 75. In addition, the permeated water separated by the reverse osmosis membrane 42 is discharged to the post-treatment filter 43.

The two-way solenoid valve 74 is a device that opens and closes a valve using the electromagnetic force of an electromagnetic coil. The two-way solenoid valve 74 has a structure in which the valve is open in a normal state, and which closes the valve in response to a signal from the controller 60.

The pressure regulating valve 73 regulates a flow rate or pressure of the concentrated water that is supplied to the intermediate tank 44.

The two-way solenoid valve 75 is a device that opens and closes a valve using the electromagnetic force of an electromagnetic coil. The two-way solenoid valve 75 has a structure in which the valve is closed in a normal state, and which opens the valve in response to a signal from the controller 60.

A sensor section 61 is arranged at a preceding stage to the reverse osmosis membrane 42. In the illustrated example, the sensor section 61 has a pressure sensor, a flow rate sensor, and an EC/temperature sensor.

The pressure sensor detects a pressure of the water supplied to the reverse osmosis membrane 42.

The flow rate sensor detects a flow rate of the water supplied to the reverse osmosis membrane 42.

The EC/temperature sensor detects the electric conductivity and temperature of the water supplied to the reverse osmosis membrane 42.

Note that, besides the aforementioned sensors, the sensor section 61 may also have a sensor that senses at least one of the items listed below.
(1) pH, oxidation-reduction potential, alkalinity, ion concentration, hardness
(2) Turbidity, chromaticity, viscosity, dissolved oxygen
(3) Odor, ammonia nitrogen, nitrate nitrogen, nitrite nitrogen, total nitrogen, residual chlorine, total phosphorus, total organic carbon, total inorganic carbon, total trihalomethane
(4) A detection result of a microbial sensor, chemical oxygen demand, biological oxygen demand
(5) Cyanogen, mercury, an oil content, surfactants
(6) A detection result of an optical sensor, a detection result of a TDS (total dissolved solids) sensor
(7) A mass spectrometry result, fine particles, zeta potential, surface potential

An air vent 71 for removing air in the water sent from the membrane filtration pump 47 is provided between the membrane filtration pump 47 and the sensor section 61.

The post-treatment filter 43 is arranged at a subsequent stage to the reverse osmosis membrane 42. The post-treatment filter 43 subjects the permeated water discharged from the reverse osmosis membrane 42 to a post-treatment that removes impurities which could not be completely filtered by the reverse osmosis membrane 42.

Although in the present embodiment an activated carbon filter is adopted as the post-treatment filter 43, the present disclosure is not limited thereto. For example, at least any one of a string wound filter, a sediment filter, an MF (microfiltration membrane), a UF (ultrafiltration membrane), an NF (nano-filtration membrane), a ceramic filter, an ion exchange filter, and a metal membrane may be selected. In the present embodiment, the post-treatment filter 43 is arranged outside the casing 2 as a part of the externally attached module 7.

A sensor section 62 is arranged at a preceding stage to the post-treatment filter 43. In the illustrated example, the sensor section 62 has a pressure sensor, a flow rate sensor, and an EC/temperature sensor.

The pressure sensor detects a pressure of the permeated water supplied to the post-treatment filter 43.

The flow rate sensor detects a flow rate of the permeated water supplied to the post-treatment filter 43.

The EC/temperature sensor detects the electric conductivity and temperature of the permeated water supplied to the post-treatment filter 43.

Note that, besides the aforementioned sensors, the sensor section 62 may also have a sensor that senses at least one of the items (1) to (7) listed above in the description of the sensor section 61.

The drainage tank 45 (second tank) is arranged at a subsequent stage to the two-way solenoid valve 75.

The drainage tank 45 is a tank for storing discharged water that is supplied thereto. The drainage tank 45 can be detached from the purification unit 40, and taken out through the door 3.

Concentrated water separated by the reverse osmosis membrane 42 and passed through the two-way solenoid valve 75 flows into the drainage tank 45. The drainage tank 45 stores the concentrated water that flows in.

A water level sensor is arranged in the drainage tank 45. The water level sensor detects a level of the discharged water stored in the drainage tank 45.

The water storage tank 46 (third tank) is arranged at a subsequent stage to the post-treatment filter 43. The water storage tank 46 is a tank for storing water supplied thereto.

Water subjected to post-treatment by the post-treatment filter 43 flows into the water storage tank 46. Hypochlorous acid water is added to the water that flows into the water storage tank 46. The water storage tank 46 stores the water to which the hypochlorous acid water has been added and which flows into the water storage tank 46.

A water level sensor is arranged in the water storage tank 46. The water level sensor detects a level of the water stored in the water storage tank 46.

The purification unit 40 includes a chlorine tank 67 and a chlorine pump 68.

The chlorine tank 67 is a tank for storing hypochlorous acid water. The hypochlorous acid water is generated, for example, by dissolving a hypochlorous acid tablet in water supplied to the chlorine tank 67. Alternatively, the hypochlorous acid water may be generated by dissolving salt in the water supplied to the chlorine tank 67, and electrolyzing the salt solution.

Note that, an electrolysis unit that subjects a salt solution to electrolysis to generate hypochlorous acid water may be separately provided on a downstream side of the chlorine tank 67.

A water level sensor is arranged in the chlorine tank 67. The water level sensor detects a level of the hypochlorous acid water stored in the chlorine tank 67.

The chlorine pump 68 is arranged at a subsequent stage to the chlorine tank 67. The chlorine pump 68 is operated under the control of the controller 60 to add hypochlorous acid water stored in the chlorine tank 67 to the water subjected to the post-treatment by the post-treatment filter 43.

### <Configuration of Externally Attached Module 7>

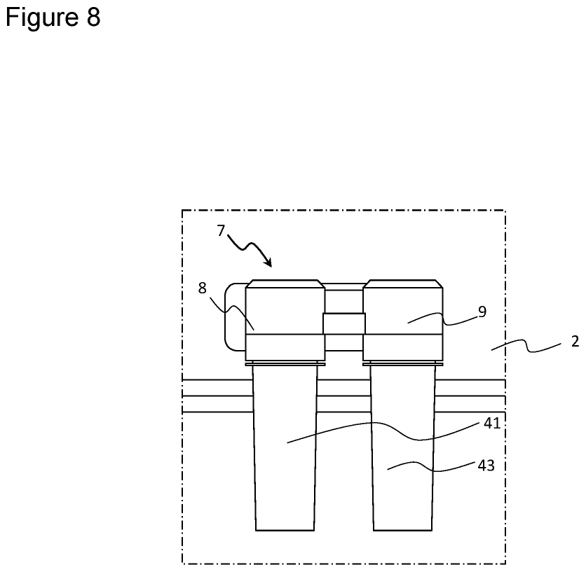

FIG. 8 is a front view of the externally attached module 7 shown in FIG. 2.

As illustrated in FIG. 8, the externally attached module 7 has the pretreatment filter 41 and the post-treatment filter 43.

The pretreatment filter 41 and the post-treatment filter 43 are arranged side by side at the exterior of the casing 2. More specifically, the pretreatment filter 41 and the post-treatment filter 43 are arranged side by side in a horizontal direction at the exterior of the casing 2.

The pretreatment filter 41 is detachably attached to an attachment section 8. The post-treatment filter 43 is detachably attached to an attachment section 9.

The pretreatment filter 41 and the post-treatment filter 43 are housed in a container having transparency whose interior is visible. Therefore, the pretreatment of water in the pretreatment filter 41, and the post-treatment of water in the post-treatment filter 43 can be visually observed from the outside.

Turbid water can be confirmed by visual observation in the pretreatment by the pretreatment filter 41. Further, it can be confirmed by visual observation that the water is purified into clean water in the post-treatment by the post-treatment filter 43.

### <Configuration of UV Sterilization Device 80>

The UV sterilization device illustrated in FIG. 5 is controlled by the controller 60.

The UV sterilization device 80 (sterilization device) has the function of irradiating ultraviolet rays at an article of a person who washes their hands with washing water, to thereby subject the surface of the article to a sterilization treatment. Articles that may be mentioned as portable items of a user include, for example, (1) an article that the user wears on their body such as eyeglasses or a wristwatch, (2) an article which the user holds in their hand, such as a key or a card, and which is used with respect to another article or device (for example, a key used to lock or unlock a door, or a card with a built-in communication chip for communicating with a payment device or the like), and (3) a device that the user operates manually, such as a smartphone.

In the present embodiment, a case where a smartphone which a user is carrying is taken as an example of a portable item is described.

The UV sterilization device 80 is provided inside the casing 2. As illustrated in FIG. 2, a hole that connects to an insertion opening 88A of the UV sterilization device 80 is formed in the top plate 10. The hole is formed at a position which is on the opposite side to the externally attached module 7, with the wash-hand basin 11 located therebetween. Here, apart from being opened in the upward direction as illustrated in the drawing, the UV sterilization device 80 may be caused to open in another direction, such as the lateral direction.

The user inserts a smartphone from the insertion opening 81.

When the smartphone is inserted from the insertion opening 81 and is placed on a pedestal (not shown) inside the UV sterilization device 80, the UV sterilization device 80 lowers the pedestal to draw the smartphone into the interior of the UV sterilization device 80.

When the pedestal is lowered to a predetermined position inside the UV sterilization device 80, a lid (not shown) is closed. When the lid has been closed, the UV sterilization device 80 turns on an ultraviolet light source (lamp).

At such time, the light source may irradiate ultraviolet rays with directivity at a predetermined region where the smartphone is likely to be particularly dirty, for example, the vicinity of the Home button, or the central region of the display. A predetermined position may be set statistically based on typical operation examples with respect to smartphones.

Further, the predetermined position may be set based on an operation log of the inserted smartphone. In this way, it is possible to shorten the time required for sterilization by irradiating ultraviolet rays in a concentrated manner at a region that is particularly dirty.

For example, the hand washing device 1 may be configured to communicate with a portable item (for example, a device having a communication function such as a smartphone) of a user to acquire information relating to the portable item or information relating to the user.

Specifically, (1) in a case where a communication section that communicates by short-range wireless communication (NFC using an IC chip or the like, or a high-speed wireless communication standard or the like) with smartphones or the like is provided in the hand washing device 1, before sterilizing the smartphone using the UV sterilization device 80 in the hand washing device 1, the user first places the smartphone at a predetermined position on the hand washing device 1. As a result, the smartphone transmits predetermined information to the hand washing device 1.

Here, a configuration may be adopted in which, as the predetermined information, the hand washing device 1 reads information indicating the smartphone model, information relating to attributes of the user of the smartphone, or the like (For example, information such as an employee code if the user is an employee or the like, or if the user is a general consumer, information indicting the age group of the user and a membership number if the user is registered with a service provided by a store or the like; information to be used when processing payments (user identification information for a payment application, credit card information or the like); or an operation log of operations the user performed on the smartphone (the coordinates of positions the user touched on the touch screen, application operation history and the like) is included. A configuration may also be adopted that allows the user to set a range of information such as these items of information to be provided to the hand washing device from the smartphone.).

In addition, (2) it shall be assumed that the hand washing device 1 is managed by an external server or the like and can communicate with the external server or the like. A two-dimensional code such as a QR code (registered trademark) that includes information identifying the hand washing device 1 (for example, a URL including identification information pertaining to the hand washing device 1) is attached to the hand washing device 1 or is displayed on a screen. A configuration may be adopted in which the smartphone of the user reads the two-dimensional code, and the smartphone transmits information including identification information pertaining to the hand washing device 1 to an external server or the like, and in response thereto the external server or the like transmits information such as the model of the smartphone to the hand washing device 1.

Further, (3) in a case where the hand washing device 1 has a reading section (an optical scanner or the like) that reads a two-dimensional code (including information indicating the model of the smartphone or the like) that is displayed on the smartphone or the like, a configuration may be adopted in which the user causes a two-dimensional code to be displayed on the screen of the smartphone and allows the hand washing device 1 to read the two-dimensional code.

Although an example in which the hand washing device 1 and the smartphone carry out wireless communication has been described above, (4) a configuration may be adopted in which the hand washing device 1 and a smartphone or the like are directly connected.

A configuration may also be adopted in which the hand washing device 1 determines ranges within which the UV sterilization device 80 is to irradiate a smartphone with ultraviolet rays based on information indicating the smartphone model. For example, a configuration may be adopted so that, in comparison to other regions, ultraviolet rays are irradiated with priority onto the position where the Home button is arranged on the smartphone (the smartphone size and the position of the Home button are defined according to the smartphone model), a region that corresponds to the lower part to the center of the screen when the user holds the smartphone vertically and operates the smartphone (a range that a finger frequently touches when the user operates the smartphone while holding the smartphone vertically), and regions corresponding to the vicinities of the left side and the right side when the user holds the smartphone horizontally and operates the smartphone. For example, in a case where the hand washing device 1 sets a time period for performing sterilization by means of the UV sterilization device 80 (time period of ultraviolet ray irradiation) to be within a certain time period in conjunction with the time period for hand washing, a configuration may be adopted so that the ranges to be subjected to ultraviolet ray irradiation by the UV sterilization device 80 with priority over other areas within the certain time period are determined as described above.

In addition, a configuration may be adopted in which the hand washing device 1 determines a hand-washing time period and a time period for which sterilization is to be performed by the UV sterilization device 80 based on information pertaining to an attribute of the user. For example, a configuration may be adopted in which, in a case where a related person such as an employee of the facility in which the hand washing device 1 is installed utilizes the hand washing device 1, the hand-washing time period and the time period for which sterilization is to be performed by the UV sterilization device 80 are set according to an attribute of the related person obtained by causing the hand washing device 1 to read information pertaining to the related person. There may be cases where hand washing and sterilization of portable items are particularly required depending on the user, such as a user whose job type is assumed to involve a lot of contact with people. By this means, the hand washing device 1 transmits a log of the times at which employees or the like washed their hands and sterilized portable items using the hand washing device 1 to an external server or the like. In a case where entry and exit to and from the facility is being managed by the external server or the like, entry and exit may be controlled based on the history of hand washing and the like performed by the user at the hand washing device 1. For example, there may be rooms that the user can enter even if the user did not wash their hands and the like at the hand washing device 1, and there may be rooms that the user cannot enter if the user did not wash their hands and the like at the hand washing device 1. By this means, the manager of the facility can restrict entry and exit to rooms based on the history of the user with respect to hand washing and the like performed by the user at the hand washing device 1.

After the UV light has been turned on for a preset time period, the UV sterilization device 80 turns off the UV light. The preset time period corresponds to, for example, a generally recommended hand-washing time period.

After turning off the UV light, the UV sterilization device 80 opens the lid and raises the pedestal to a predetermined position. The term “predetermined position” refers to a position at which a part of the smartphone protrudes from the insertion opening 81 so as to be accessible to the user.

Thus it is possible for the smartphone to be sterilized by being inserted into and taken out from the UV sterilization device 80 by the user without the user touching the UV sterilization device 80.

The hand washing device 1 may also be configured to dispense washing water from the spout 13 in response to a portable item being placed in the UV sterilization device 80. By this means, the occurrence of a situation in which the user forgets to sterilize a portable item in the UV sterilization device 80 can be prevented.

### <Control Processing of Hand Washing Device 1>

Next, control processing of the hand washing device 1 will be described. FIG. 17 is explanatory diagrams illustrating control processing of the hand washing device 1.

### (Spouting Process of Spout Unit 20)

First, a process for spouting water from the faucet 12 that is carried out by the spout unit 20 will be described.

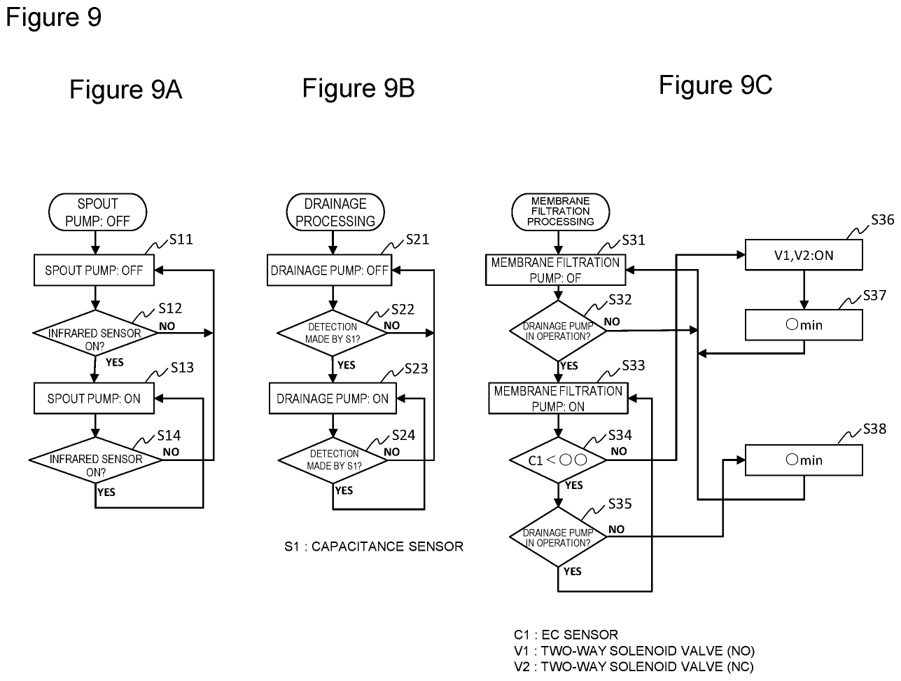

As illustrated in FIG. 9A, the controller 60 turns the spout pump 21 off (step S11).

The controller 60 determines whether or not an object has been detected by the infrared sensor 23 (step S12). When a user utilizes the hand washing device 1, the user puts their hand into the wash-hand basin 11 and causes the infrared sensor 23 arranged at the tip portion of the faucet 12 to detect one or more fingers.

In response to the detection of a finger by the infrared sensor 23 (Yes in step S12), the controller 60 actuates the spout pump 21 (step S13).

The spout pump 21 causes water stored in the water storage tank 46 to pass through the UV sterilization section 22. The UV sterilization section 22 irradiates ultraviolet rays at the water sent from the spout pump 21, to thereby subject the water to a sterilization treatment. The water that passes through the UV sterilization section 22 is spouted from the spout 13 of the faucet 12 as washing water.

On the other hand, in step S12, if a finger is not detected by the infrared sensor 23 (No in step S12), the spout pump 21 does not operate (step S11).

The controller 60 determines whether or not an object is detected by the infrared sensor 23 (step S14). When the user finishes utilizing the hand washing device 1, or when the user causes the dispenser 14 to dispense a pharmaceutical agent, the user moves their fingers away from the infrared sensor 23 arranged at the tip portion of the faucet 12.

In response to the non-detection of a finger by the infrared sensor 23 (No in step S14), the controller 60 stops the spout pump 21 (step S11).

On the other hand, in step S14, if non-detection of a finger by the infrared sensor 23 does not occur (Yes in step S14), the controller 60 continues to operate the spout pump 21 (step S13).

The spout unit 20 repeats these processes and spouts washing water from the faucet 12.

The controller 60 may be configured to measure a time period for which a finger is detected by the infrared sensor 23, and to stop water being spouted from the faucet 12 if a finger has been detected for a certain period of time.

### (Drainage Process of Drainage Unit 30)

Next, a drainage process performed by the drainage unit 30 will be described.

As illustrated in FIG. 9B, in a state in which no water is being drained from the wash-hand basin 11, the drainage pump 32 is stopped (step S21).

Subsequently, upon washing water spouted from the faucet 12 toward the wash-hand basin 11 being drained from the drainage outlet 17 of the wash-hand basin 11, the capacitance sensor 31 detects the drained water (step S22).

When the capacitance sensor 31 detects the drained water (Yes in step S22), the controller 60 actuates the drainage pump 32 (step S23).

On the other hand, if the capacitance sensor 31 does not detect drained water (No in step S22), the controller 60 does not actuate the drainage pump 32 (step S21).

In step S23 the drainage pump 32 sends the drained water to the pretreatment filter 41. Water subjected to a pretreatment by the pretreatment filter 41 flows into the intermediate tank 44 and is stored in the intermediate tank 44. The pressure sensor 33 detects the pressure of the water sent to the pretreatment filter 41. The flow rate sensor 34 detects the flow rate of the water subjected to the pretreatment by the pretreatment filter 41.

Subsequently, after step S23, if the capacitance sensor 31 detects that drained water has stopped flowing in from the drainage outlet 17 (No in step S24), the controller 60 stops the drainage pump 32 (step S21).

On the other hand, in step S24, if the capacitance sensor 31 detects that drained water continues to flow in from the drainage outlet 17 (Yes in step S24), the controller 60 continues to operate the drainage pump 32 (step S23).

The drainage unit 30 repeats these processes to supply water that is drained from the drainage outlet 17 of the wash-hand basin 11 to the pretreatment filter 41.

### (Control Process of Purification Unit 40)

Next, a purification process performed by the purification unit 40 will be described.

As illustrated in FIG. 9C, initially, the membrane filtration pump 47 is stopped (step S31).

The controller 60 determines whether or not the drainage pump 32 is being operated (step S32).

If the drainage pump 32 is operating (Yes in step S32), the controller 60 actuates the membrane filtration pump 47 (step S33). By this means, water stored in the intermediate tank 44 is supplied at high pressure to the reverse osmosis membrane 42 by the membrane filtration pump 47.

On the other hand, if the drainage pump 32 is not operating (No in step S32), the controller 60 maintains a state in which the membrane filtration pump 47 is not actuated (step S31).

In step S33, the water supplied to the reverse osmosis membrane 42 is separated into concentrated water and permeated water by the reverse osmosis membrane 42. The permeated water is supplied to the post-treatment filter 43.

The concentrated water flows into the intermediate tank 44 via the two-way solenoid valve 74. Note that, because the two-way solenoid valve 75 is in a closed state, a situation does not occur in which the concentrated water flows into the drainage tank 45.

At the post-treatment filter 43, the permeated water is subjected to a post-treatment. Then, hypochlorous acid water is added to the permeated water subjected to the post-treatment by the post-treatment filter 43, and the permeated water to which the hypochlorous acid water has been added flows into the water storage tank 46.

After step S33, the controller 60 determines whether or not the electric conductivity detected by the EC/temperature sensor of the sensor section 61 arranged at a preceding stage to the reverse osmosis membrane 42 is less than a predetermined value (step S34).

If the electric conductivity detected by the EC/temperature sensor is less than the predetermined value (Yes in S34), the controller 60 determines whether or not the drainage pump 32 is operating (step S35). If the drainage pump 32 is operating (Yes in step S35), the controller 60 continues to operate the membrane filtration pump 47 (step S33).

On the other hand, in step S34, if the electric conductivity detected by the EC/temperature sensor is equal to or greater than the predetermined value (No in step S34), the controller 60 turns the two-way solenoid valve 74 and the two-way solenoid valve 75 on (step S36). As a result, the two-way solenoid valve 74 is closed, and the two-way solenoid valve 75 is opened.

By the two-way solenoid valve 74 being closed and the two-way solenoid valve 75 being opened, the concentrated water separated by the reverse osmosis membrane 42 flows into the drainage tank 45 via the two-way solenoid valve 75. That is, the controller 60 determines an amount of impurities in the concentrated water based on a change in electrical conductivity detected by the EC/temperature sensor. Concentrated water that is determined as containing a large amount of impurities is discharged to the drainage tank 45.

When the water level of the concentrated water stored in the drainage tank 45 reaches a predetermined value, for example, an alert is issued to a manager of the hand washing device 1. When the manager confirms the alert, the water stored in the drainage tank 45 is discarded.

The controller 60 continues the process in step S36 for a predetermined period of time (step S37), and after the predetermined period of time has elapsed, the controller 60 turns the two-way solenoid valves 74 and 75 off. As a result, the two-way solenoid valve 74 is opened and the two-way solenoid valve 75 is closed.

Thus, by the two-way solenoid valve 74 being opened and the two-way solenoid valve 75 being closed, concentrated water separated by the reverse osmosis membrane 42 flows into the intermediate tank 44 via the two-way solenoid valve 74. The controller 60 then stops the membrane filtration pump 47 (step S31).

In step S35, if the drainage pump 32 is stopped (No in step S35), after a predetermined period of time has elapsed (step S38), the controller 60 stops the membrane filtration pump 47 (step S31).

The purification unit 40 repeats these processes to thereby purify water drained by the drainage unit 30 and store the purified water in the water storage tank 46.

(Control processing and display modes of hand-washing indicator 15)

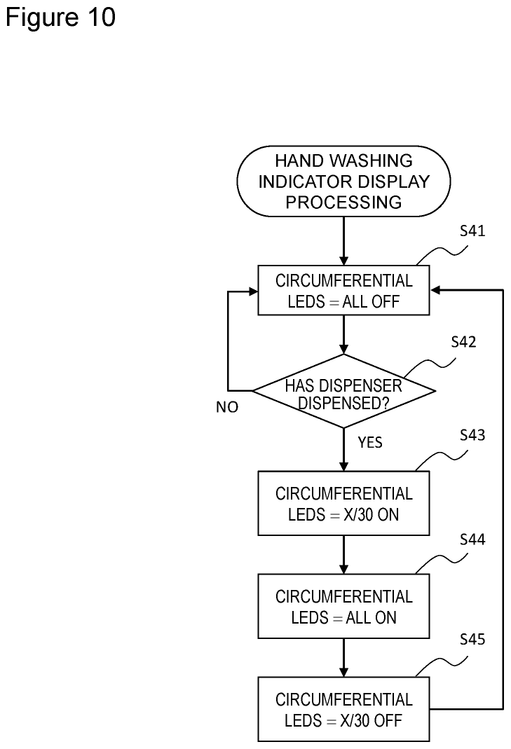

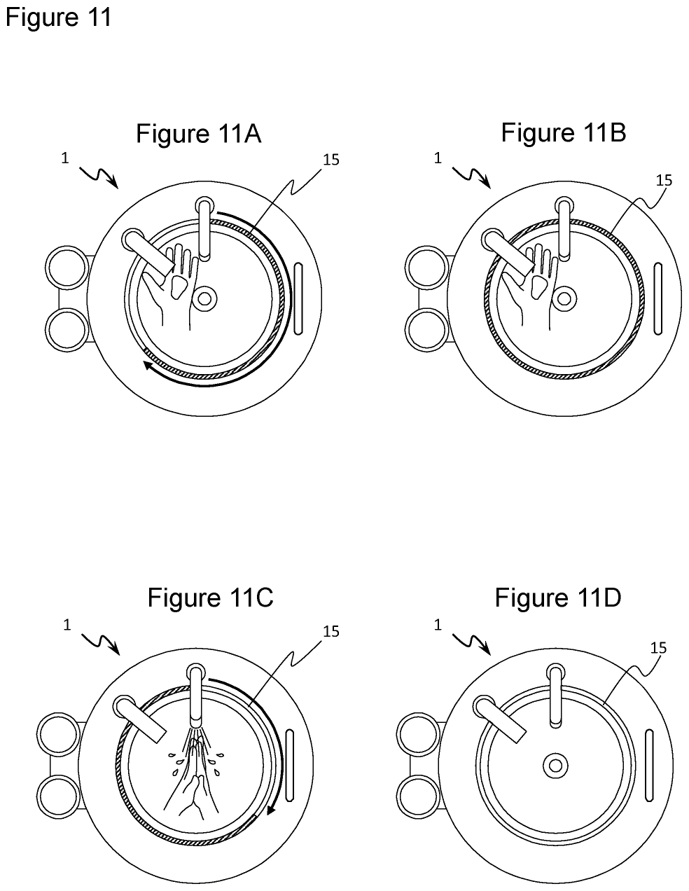

Next, display modes and control processing for the hand-washing indicator 15 will be described. FIG. 10 is an explanatory drawing illustrating control processing for the hand-washing indicator 15. FIG. 11 is views for describing display modes of the hand-washing indicator 15.

As illustrated in FIG. 10, when the hand washing device 1 is not in use, the LED lights constituting the hand-washing indicator 15 are turned off (step S41).

Next, the controller 60 determines whether or not the dispenser 14 operated (step S42). When a user who uses the hand washing device 1 holds their hands below the dispenser 14, the infrared sensor 52 of the dispenser 14 detects one or more fingers. Upon one or more fingers being detected by the infrared sensor 52, the controller 60 actuates the pharmaceutical agent pump 51 to cause the pharmaceutical agent to be dispensed from the dispenser 14.

If the dispenser 14 operated (Yes in step S42), the controller 60 causes the hand-washing indicator 15 to light up (step S43 to step S44).

Specifically, as illustrated in FIG. 11A, the controller 60 causes the 30 LED lights constituting the hand-washing indicator 15 to light up in clockwise order, starting from the LED light positioned in the vicinity of the base of the faucet 12 in top view. At such time, the controller 60 controls so that, for example, all of the LED lights light up for about 1 to 2 seconds (step S43 in FIG. 10).

Further, as illustrated in FIG. 11B, when all of the LED lights have lit up (step S44 in FIG. 10), the controller 60 maintains the lighting state for a predetermined time period thereafter. After the predetermined time period elapses, as illustrated in FIG. 11C, the control unit 60 turns off the LED lights in clockwise order, starting from the LED light positioned in the vicinity of the base of the faucet 12 (step S45 in FIG. 10).

At such time, the controller 60 controls so that, for example, all of the LED lights turn off within a period of about 30 to 40 seconds.

The hand washing device 1 may be configured so that, in response to water being dispensed from the spout 13 when one or more fingers of a user are detected by the infrared sensor 23, the lights of the hand-washing indicator 15 are turned off sequentially as time passes in the manner described above. By this means, because the lighting state of the hand-washing indicator 15 changes as water is being dispensed from the spout 13, the user can be made aware of the time period from when dispensing of the water begins until the hand washing is to be completed.

Further, the hand washing device 1 may be configured so that, in a case where one or more fingers of a user are not detected by the infrared sensor 23, the hand-washing indicator 15 is caused to light up in a predetermined manner (caused to emit light in a predetermined color or the like) or the like to notify users in the area around the hand washing device 1 to urge the users to wash their hands.

Further, as illustrated in FIG. 11D, when all of the LED lights have turned off (step S45 in FIG. 10), the hand-washing indicator 15 returns to the initial state (step S41).

The time period until the LED lights of the hand-washing indicator 15 turn off sequentially after lighting up sequentially is set to a generally recommended hand-washing time period, for example, about 40 seconds. By this means, the hand washing device 1 can provide the user with a guideline regarding a hand-washing time period with respect to which a sufficient cleaning effect can be expected.

As described above, the antenna 56 is provided inside the nozzle 53 of the dispenser 14. By this means, the dispenser 14 can maintain an elegant appearance while being equipped with communication means for communicating with an external device.

Further, the antenna 56 is arranged along the extending direction of the pipe 55 that forms a channel inside the nozzle 53. Thus, by housing the antenna 56 in a manner that effectively utilizes the internal space of the nozzle 53 as much as possible, it is possible to prevent the nozzle 53 from being extremely large.

In addition, the nozzle 53 is made of a resin material. Therefore, the antenna 56 housed inside the nozzle 53 can smoothly transmit and receive radio waves.

Further, the pharmaceutical agent pump 51 discharges a pharmaceutical agent from the pharmaceutical agent tank 50 that is housed in the casing 2 which is made of metal. Therefore, unlike a configuration in which, for instance, the antenna 56 is housed inside the casing 2, the occurrence of a situation in which the casing 2 made of metal interferes with transmission and reception of radio waves to and from the outside by the antenna 56 can be suppressed.

Further, since the hand washing device 1 includes the dispenser 14 in which the antenna 56 is housed inside the nozzle 53, the hand washing device 1 can maintain an elegant appearance while being equipped with communication means for transmitting and receiving various kinds of data relating to operation of the hand washing device 1 to and from an external device.

### <Modification>

Next, a modification of the dispensing device according to the present disclosure will be described. Note that, the same reference characters are assigned to those components that are the same as the corresponding components in the above embodiment, and a description of such components is omitted below.

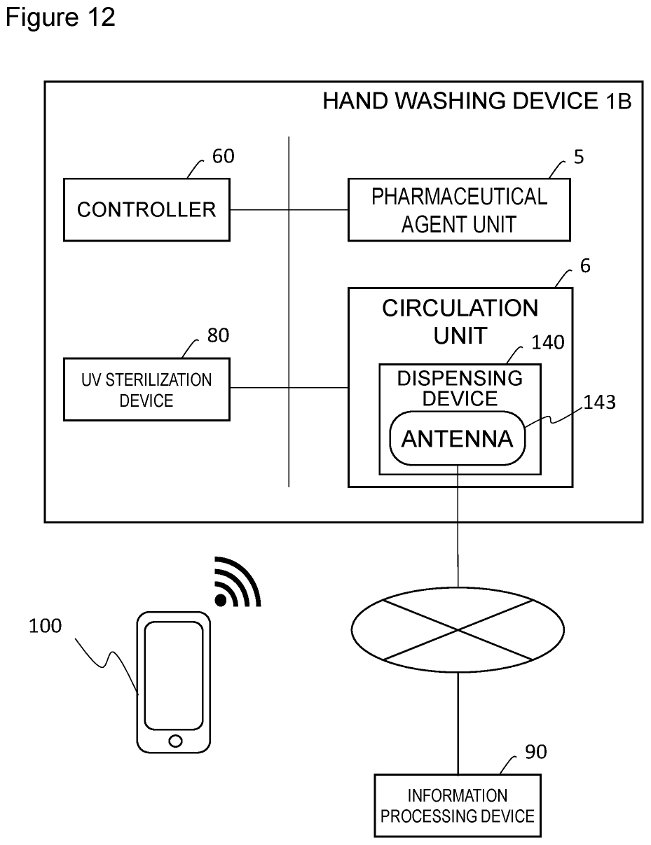

In this modification, a configuration of a dispensing device that dispenses water will be described. FIG. 12 is a block diagram of a hand washing device 1B in which a dispensing device 140 according to the present modification is mounted.

In the hand washing device 1B, the circulation unit 6 includes the dispensing device 140 having an antenna 143. Antennas that are compatible with different standards to each other may be provided in the dispensing device 140 and the pharmaceutical agent unit 5, respectively.

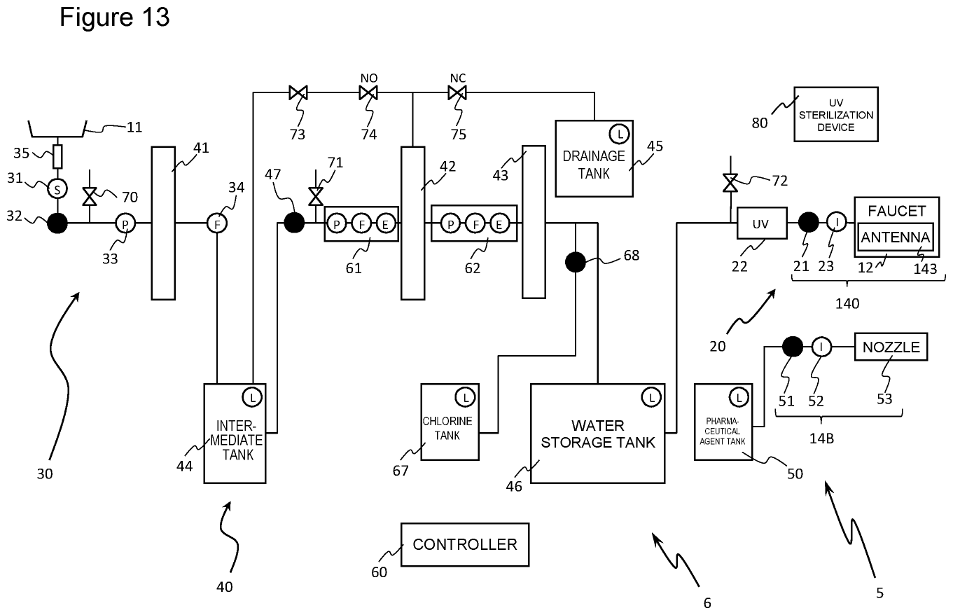

FIG. 13 is a block diagram for describing each unit of the hand washing device 1B. As illustrated in FIG. 13, in the circulation unit 6, the spout unit 20 includes a UV sterilization section 22 and the dispensing device 140.

The dispensing device 140 includes the spout pump 21, the infrared sensor 23, and the faucet (nozzle 12).

The spout pump is arranged at a subsequent stage to the UV sterilization section 22, and sends water stored in the water storage tank 46 toward the faucet 12.

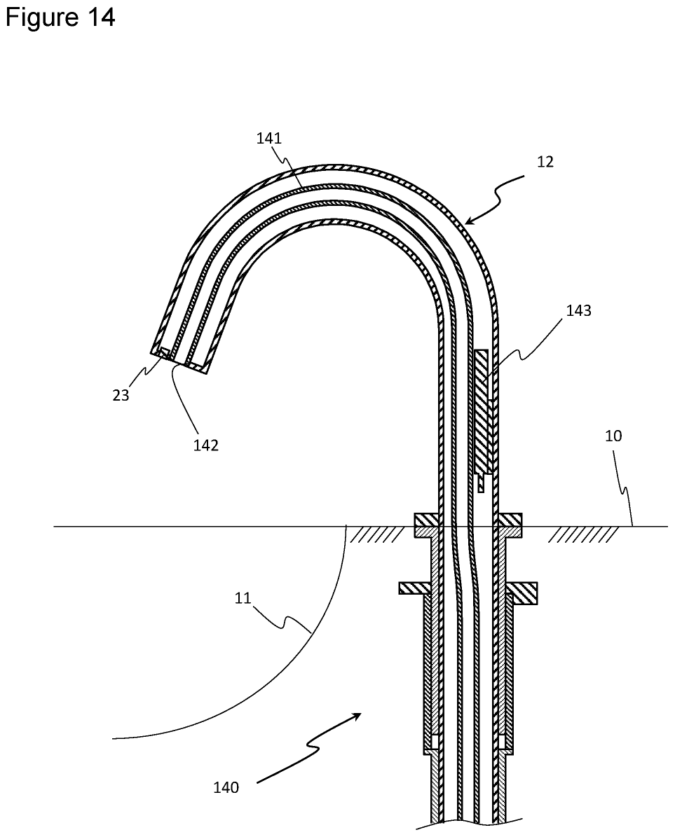

FIG. 14 is a cross-sectional view of the dispensing device 140. As illustrated in FIG. 14, the infrared sensor 23, a pipe 141, a dispensing portion 142, and the antenna 143 are provided in the faucet 12. The faucet 12 is made of a synthetic resin material.

The infrared sensor 23 is arranged at the tip of the faucet 12.

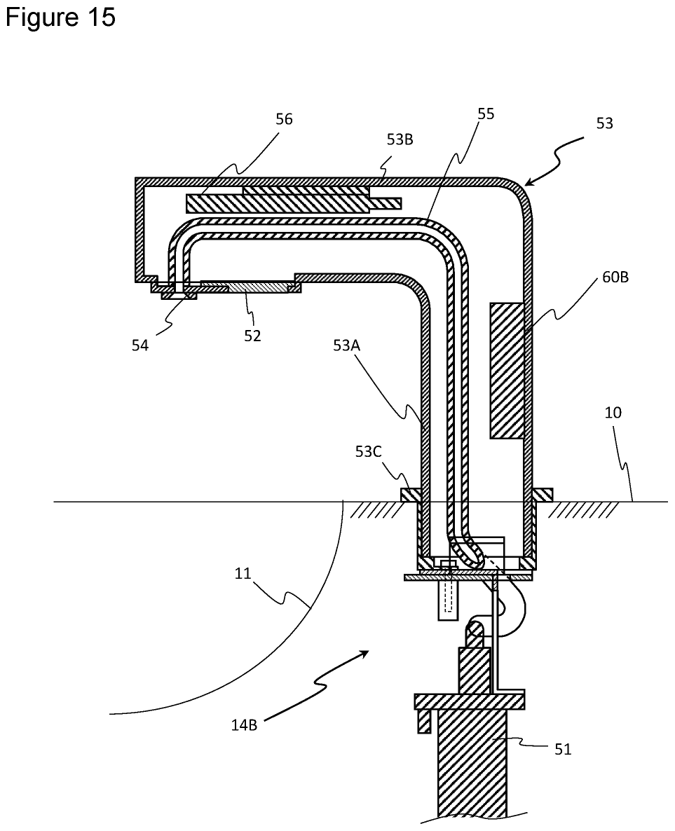

The pipe 141 is connected to the spout pump 21 which is not shown in FIG. 15, and allows water that is sent by the spout pump 21 to flow therein.

The dispensing portion 142 is provided at the tip of the faucet 12, and dispenses water that flows down through the pipe 141, into the inside of the wash-hand basin 11.

The antenna 143 is provided inside the nozzle, and transmits and receives radio signals. The antenna 143 is arranged along the direction in which the pipe 141 is extended. Similarly to the above embodiment, the antenna 143 transmits radio signals relating to various types of information to an external information processing device, and receives such radio signals from the information processing device.

The antenna 143 is, for example, an antenna that is capable of receiving radio waves in accordance with at least any one communication standard. Further, the antenna 143 is an antenna that is capable of transmitting radio waves in accordance with at least any one communication standard.

For example, the antenna 143 may be compatible with a communication service provided by a telecommunications carrier. In such a case, for example, a communication function realized by the controller 60 of the hand washing device 1 is compatible with the communication service that the telecommunications carrier provides. By this means it is possible for the hand washing device 1 to be connected via the antenna 143 to a network which the telecommunications carrier provides.

Further, for example, the antenna 143 may be compatible with a short-range wireless communication technology such as Bluetooth (registered trademark), ZigBee (registered trademark), or BLE (Bluetooth Low Energy). In such a case, for example, a communication function realized by the controller 60 of the hand washing device 1 performs communication processing that corresponds to the short-range wireless communication such as Bluetooth (registered trademark), ZigBee (registered trademark), or BLE (Bluetooth Low Energy). By this means it is possible for the hand washing device 1 to be communicably connected via the antenna 143 to a portable item (a communication device such as a smartphone or a smartwatch) that supports the short-range wireless communication.

In addition, for example, the antenna 143 may be compatible with a short-range wireless communication technology such as NFC (near field communication). In such a case, for example, a communication function realized by the controller 60 of the hand washing device 1 performs communication processing that corresponds to the short-range wireless communication such as NFC. By this means it is possible for the hand washing device 1 to read out data from a portable item that supports short-range wireless communication via the antenna 143.

Data which the hand washing device 1B transmits or receives by means of the antenna 143 is the same as in the above embodiment.

### <Other Modification>

A dispenser 14B that is a dispensing device according to another modification will now be described. FIG. 15 is a cross-sectional view of the dispenser 14B.

In the dispenser 14B, the antenna 56 is housed in the arm portion 53B of the nozzle 53. In this way, the position where the antenna 56 is housed can be arbitrarily changed.

Further, a communication section 60B that realizes a communication function of the controller 60 is housed inside the base 53A of the nozzle 53 in the dispenser 14B. The communication section 60B is realized, for example, by a processor, a memory, and a storage. The communication section 60B is connected to the antenna 56.

For example, the following data is stored in the storage.
Data relating to hand washing
Data relating to maintenance
Data input from portable item
Data transmitted to portable item

Thus, in the dispenser 14B, the communication section 60B is housed inside the nozzle 53. Therefore, the components arranged inside the casing 2 of the hand washing device 1 decrease, and hence it is possible to leave a margin with respect to the space inside the casing 2.

Note that, the communication section 60B may be provided inside the dispensing device 140 illustrated in FIG. 14, similarly to the dispenser 14B.

Although a preferred embodiment of the present disclosure has been described above, the present disclosure is not limited to such a specific embodiment, and the present disclosure encompasses the invention described in the claims as well as equivalents thereof. In addition, some of the configurations of the devices described in the above embodiment and the modifications thereof can be omitted or combined as long as no technical contradiction arises.

The matters described in the above embodiment will be added below.

### APPENDIX 1

Dispensing devices 14 and 140, including: pumps 51 and 21 which discharge a liquid, pipes 55 and 141 through which a liquid discharged by the pumps 51 and 21 flows, dispensing portions 54 and 142 which are connected to the pipes 55 and 141 and which dispense a liquid that flows through the pipes 55 and 141, nozzles 53 and 12 that house the pipes 55 and 141 and in which the dispensing portions 54 and 142 are arranged in opening portions, and antennas 56 and 143 which are provided inside the nozzles 53 and 12 and which transmit and receive radio signals.

### APPENDIX 2

The dispensing devices 14 and 140 according to (Appendix 1), wherein the antennas 56 and 143 are arranged along extending directions of the pipes 55 and 141.

### APPENDIX 3

The dispensing devices 14 and 140 according to (Appendix 1) or (Appendix 2), wherein the nozzles 53 and 12 are made of a resin material.

### APPENDIX 4

The dispensing devices 14 and 140 according to any one of (Appendix 1) to (Appendix 3), wherein the pumps 51 and 21 discharge water from a tank 46 housed in a casing 2 made of metal.

### APPENDIX 5

The dispensing devices 14 and 140 according to any one of (Appendix 1) to (Appendix 3), wherein the pumps 51 and 21 discharge a pharmaceutical agent from a tank 50 housed in a casing 2 made of metal.

### APPENDIX 6

Hand washing devices 1 and 1B, including: pumps 51 and 21 which discharge a liquid, pipes 55 and 141 through which a liquid discharged by the pumps 51 and 21 flows, dispensing portions 54 and 142 which are connected to the pipes 55 and 141 and which dispense a liquid that flows through the pipes 55 and 141, a wash-hand basin 11, nozzles 53 and 12 that house the pipes 55 and 141, and in which the dispensing portions 54 and 142 are arranged in opening portions that are formed above the wash-hand basin 11, antennas 56 and 143 which are provided inside the nozzles 53 and 12 and which transmit and receive radio signals, tanks 46 and 50 which supply a liquid to the pumps 51 and 21, and a circulation unit (a circulation mechanism) 6 which circulates and purifies drained water from the wash-hand basin 11.

1. A dispensing device comprising:
a pump that discharges a liquid,
a pipe through which a liquid discharged by the pump flows,
a dispensing portion that is connected to the pipe and dispenses a liquid that flows through the pipe,
a nozzle that houses the pipe, the dispensing portion being arranged therein in an opening portion, and
an antenna that is provided inside the nozzle and transmits and receives radio signals.

2. The dispensing device according to claim 1, wherein:
the antenna is arranged along an extending direction of the pipe.

3. The dispensing device according to claim 1, wherein:
the nozzle is formed of a resin material.

4. The dispensing device according to claim 1, wherein:
the pump discharges water from a tank housed in a casing made of metal.

5. The dispensing device according to claim 3, wherein:
the pump discharges water from a tank housed in a casing made of metal.

6. The dispensing device according to claim 1, wherein:
the pump discharges a pharmaceutical agent from a tank housed in a casing made of metal.

7. The dispensing device according to claim 3, wherein:
the pump discharges a pharmaceutical agent from a tank housed in a casing made of metal.

8. A hand washing device comprising:
a pump that discharges a liquid,
a pipe through which a liquid discharged by the pump flows,
a dispensing portion that is connected to the pipe and dispenses a liquid that flows through the pipe,
a wash-hand basin,
a nozzle that houses the pipe, the dispensing portion being arranged therein in an opening portion that is formed above the wash-hand basin,
an antenna that is provided inside the nozzle and transmits and receives radio signals,
a tank that supplies a liquid to the pump,
a circulation mechanism that circulates and purifies drained water from the wash-hand basin.
